(19) United States
(12) Patent Application Publication
(10) Pub. No.: US 2013/0288311 A1
(43) Pub. Date: Oct. 31, 2013

(54) CELLULOSE SACCHARIFICATION APPARATUS, BIOMASS SACCHARIFICATION APPARATUS, FERMENTATION APPARATUS AND CELLULOSE SACCHARIFICATION METHOD

(71) Applicants: Kenji Sato, Tokyo (JP); Makoto Kitano, Tokyo (JP); Tatsuya Oka, Tokyo (JP); Kentaro Nariai, Tokyo (JP); Norimitsu Kaneko, Tokyo (JP); Michikazu Hara, Yokohama-shi (JP)

(72) Inventors: Kenji Sato, Tokyo (JP); Makoto Kitano, Tokyo (JP); Tatsuya Oka, Tokyo (JP); Kentaro Nariai, Tokyo (JP); Norimitsu Kaneko, Tokyo (JP); Michikazu Hara, Yokohama-shi (JP)

(21) Appl. No.: 13/876,264

(22) PCT Filed: Sep. 27, 2011
(86) PCT No.: PCT/JP2011/072713

§ 371 Date: May 30, 2013

(30) Foreign Application Priority Data
Sep. 27, 2010 (JP) 2010-216134
Mar. 10, 2011 (JP) 2011-053503

(51) Int. Cl. C12M 1/00; C12P 19/02

(52) U.S. Cl. CPC C12M 43/00; C12P 19/02
USPC 435/105; 435/289.1

(57) ABSTRACT

A fermentation apparatus (A) of the present invention comprising: an enzymatic reactor (4) for degrading cellulose using a diastatic enzyme, and a first catalytic reactor (5) for degrading the degradation product produced by the enzymatic reactor (4) into glucose, using a solid acid catalyst (X). According to this fermentation apparatus (A), saccharification treatment of cellulose can be performed while reducing diastatic enzyme costs.

### TECHNICAL FIELD

The present invention relates to a cellulose saccharification apparatus, a biomass saccharification apparatus, a fermentation apparatus and a cellulose saccharification method. Priority is claimed on Japanese Patent Application No. 2010-216134, filed Sep. 27, 2010, and Japanese Patent Application No. 2011-53503, filed Mar. 10, 2011. The contents of which are incorporated herein by reference.

### BACKGROUND ART

As techniques for producing ethanol (bioethanol) from biomass, a variety of processes has been published. For example, the following Non-Patent Document 1 discloses a process for producing ethanol, involving saccharification of cellulose in biomass into glucose, using cellulase which is widely known as a diastatic enzyme, and fermentation of the glucose. In the saccharification of cellulose using the diastatic enzyme, cellulose is degraded into cellobiose (glucose dimer) through the action of β-glucanase contained in the diastatic enzyme, and similarly the cellobiose is finally degraded into glucose through the action of β-glucosidase contained in the diastatic enzyme.

### PRIOR ART DOCUMENT

### Patent Document

[Non-Patent Document 1] Koreishi Mayuko, Imanaka Hiroyuki, Imamura Koreyoshi, Kariyama Masahiro, and Nakanishi Kazuhiro, “Efficient Ethanol Production from Wheat Bran by Enzymatic Saccharification Using Commercially Available Enzyme Products and Fermentation Using Bakers' Yeast”, The Society for Biotechnology, Japan, Vol. 87 (5), P. 216-223, 2009.

### DISCLOSURE OF THE INVENTION

### Problem that the Invention is to Solve

However, in case of the above-stated conventional art, running costs are increased due to increased amounts of diastatic enzyme used, since the diastatic enzyme undergoes deactivation over time. For example, it is estimated that the diastatic enzyme accounts for 20% or more of the production costs for the production of ethanol from biomass.

Further, since β-glucosidase contained in the diastatic enzyme is inhibited by glucose which is finally produced through the saccharification treatment, in case of the above-stated conventional art, the hydrolysis rate of cellobiose decreases in response to the production of glucose, which consequently results in lowering of the glucose production rate. In order to address these problems, further addition of β-glucosidase is needed, which, in turn, leads to an increase in running costs.

Therefore, the present invention has been made in view of the above situation, and is intended to provide the following objects.

(1) Saccharification of cellulose or biomass in conjunction with further reduction of diastatic enzyme costs as compared to the conventional art.

(2) Production of fermentative products from biomass in conjunction with further reduction of diastatic enzyme costs as compared to the conventional art.

### Means for Solving the Problem

In order to achieve the above-stated objects, a cellulose saccharification apparatus according to the present invention includes an enzymatic reactor for the degradation of cellulose into cellobiose using a diastatic enzyme, and a first catalytic reactor for the degradation product of cellobiose produced by the enzymatic reactor into glucose, using a solid acid catalyst.

In this cellulose saccharification apparatus, it is preferable that the diastatic enzyme is a heat-resistant enzyme.

Further, a biomass saccharification apparatus according to the present invention includes a pressurized hot water reactor for selective degradation of hemicellulose contained in biomass by allowing pressurized hot water to act on the biomass, a solid-liquid separator for the separation of cellulose as a solid from a treated liquid of the pressurized hot water reactor, and a cellulose saccharification apparatus in accordance with the first or second solution means for the degradation of cellulose separated by the solid-liquid separator into glucose.

In the present invention the biomass saccharification apparatus may further includes a second catalytic reactor for the degradation of a hemicellulose degradation product as the liquid separated by the solid-liquid separator, into a hemicellulose-derived monosaccharide, using a solid acid catalyst.

A fermentation apparatus according to the present invention includes a biomass saccharification apparatus having the second catalytic reactor, a first fermenter for the production of fermentative products from glucose produced by the biomass saccharification apparatus, and a second fermenter for the production of fermentative products from a hemicellulose-derived monosaccharide produced by the biomass saccharification apparatus.

A cellulose saccharification method according to the present invention includes an enzymatic reaction process for the degradation of cellulose into cellobiose using a diastatic enzyme, and a solid acid catalytic reaction process for the degradation product produced by the enzymatic reaction process into glucose, using a solid acid catalyst.

### Advantage of the Invention

In the present invention, cellulose is degraded by an enzymatic reaction based on a diastatic enzyme, and further, the degradation product produced by the enzymatic reaction is degraded into glucose by a catalytic reaction based on a solid acid catalyst. That is, the conventional art exhibits an increase in diastatic enzyme costs because the above-stated two degradation processes are implemented by an enzymatic reaction incapable of being reused, whereas the present invention can perform saccharification treatment of cellulose while reducing diastatic enzyme costs, since the degradation product produced by the enzymatic reaction into glucose is carried out using a reusable solid acid catalyst in place of diastatic enzyme costs. Accordingly, diastatic enzyme costs can be further reduced as compared to the conventional art, even when fermentable sugar such as glucose is produced from cellulose contained in biomass, and further even when fermentable sugar such as glucose is produced from cellulose contained in biomass and fermentative products such as ethanol is produced from the fermentable sugar.

### BRIEF DESCRIPTION OF THE DRAWINGS

FIG. 1 shows a process block diagram of an ethanol production apparatus in accordance with an embodiment of the present invention.

FIG. 2A shows the simulation results representing a saccharide concentration upon performing an enzymatic reaction in ethanol production apparatus in accordance with an embodiment of the present invention.

FIG. 2B shows the simulation results representing a saccharide concentration upon performing a solid acid catalytic reaction, in ethanol production apparatus in accordance with an embodiment of the present invention.

FIG. 3A is a bar diagram which shows concentration of degradation products in accordance with an embodiment of the present invention based on experimental results.

FIG. 3B is a line graph which shows temperature dependency of a production rate constant of glucose and a decomposition constant of glucose of a solid acid catalyst in accordance with an embodiment of the present invention based on experimental results.

### BEST MODE FOR CARRYING OUT THE INVENTION

Hereinafter, embodiments of the present invention will be described with reference to the accompanying drawings.

An ethanol production apparatus (fermentation apparatus) A in accordance with this embodiment includes a pressurized hot water reactor 1, a solid-liquid separator 2, a cooler 3, an enzymatic reactor 4, a first catalytic reactor 5, a first fermenter 6, a second catalytic reactor 7, a second fermenter 8, a distillation unit 9, and a drainage unit 10. The ethanol production apparatus A performs a process that produces monosaccharides (xylose and glucose) by subjecting ligneous biomass externally supplied as a raw material to saccharification treatment, and produces high-purity ethanol by subjecting the monosaccharides to alcohol fermentation and distillation treatments.

The pressurized hot water reactor 1 performs selective hydrolysis and solubilization of hemicellulose (solid) contained in ligneous biomass through the action of pressurized hot water of 150 to 230° C., more preferably 200 to 230° C. on the ligneous biomass. The ligneous biomass is a cellulose-based biomass containing cellulose, hemicellulose and lignin as main components. When pressurized hot water of a relatively low temperature of 150 to 230° C. is applied, hemicellulose, among these main components, is easily hydrolyzed, that is, it is degraded (solubilized) into a polysaccharide (hemicellulose degradation product) whose main component is a hemicellulose-derived oligosaccharide in which pentoses are polymerized, whereas cellulose is substantially not degraded in pressurized hot water of 200° C. or so. In particular, for hydrolysis of cellulose with pressurized hot water, action of pressurized hot water with a higher temperature than 200° C., for example, 240 to 300° C., is needed on ligneous biomass.

The pressurized hot water reactor 1 performs selective hydrolysis of hemicellulose contained in ligneous biomass into a polysaccharide (hemicellulose degradation product) whose main component is a hemicellulose-derived oligosaccharide in which pentoses are polymerized, by taking advantage of such characteristics of cellulose, hemicellulose and lignin in response to pressurized hot water. As used herein, the term “pressurized hot water” refers to subcritical-state hot water and is hot water pressurized to maintain a liquid state.

The pressurized hot water reactor 1 includes, as shown in the drawing, a pump 1a, a heater 1b, a water volume control valve 1c, a reaction bath 1d, and a control unit 1e. The pump 1a pressurizes water supplied from the outside, and delivers the pressurized water to the heater 1b. The heater 1b heats the pressurized water coming from the pump 1a to a temperature of 150 to 230° C., more preferably 200 to 230° C., in response to temperature control signals being input from the control unit 1e, and delivers the pressurized hot water to the water volume control valve 1c. The water volume control valve 1c is an electronic control valve, the opening of which is controlled in response to flow rate control signals input from the control unit 1e, and adjusts the flow rate and then delivers pressurized hot water coming from the heater 1b to the reaction bath 1d.

The reaction bath 1d receives a given amount of ligneous biomass supplied as a raw material from the outside, and selectively degrades hemicellulose in the ligneous biomass into a polysaccharide containing a hemicellulose-derived oligosaccharide as a main component, by the addition (action) of pressurized hot water coming from the water volume control valve 1c to the ligneous biomass. That is, the treated liquid of the reaction bath 1d contains cellulose and lignin as solids, among main components of the ligneous biomass, and also contains a polysaccharide (hemicellulose degradation product) as a liquid, which contains a hemicellulose-derived oligosaccharide obtained by degradation of hemicellulose as a main component. The reaction bath 1d discharges such a treated liquid to the solid-liquid separator 2.

The control unit 1e outputs temperature control signals to the heater 1b, outputs flow rate control signals to the water volume control valve 1c, and controls hydrolysis conditions of the ligneous biomass present in the reaction bath 1d by controlling the temperature and flow rate (supply amount) of pressurized hot water supplied to the reaction bath 1d. That is, the control unit 1e sets a ratio K(=Q/V) of a supply amount Q (ml) of pressurized hot water and a supply amount V (g) of ligneous biomass, and a temperature T (° C.) of pressurized hot water, as hydrolysis conditions. By controlling the hydrolysis conditions of the reaction bath 1d by means of the control unit 1e, the treated liquid discharged from the reaction bath 1d contains, as described above, cellulose and lignin as a solid, and also contains, as a liquid, a polysaccharide containing a hemicellulose-derived oligosaccharide obtained by degradation of hemicellulose as a main component.

The solid-liquid separator 2 delivers solid cellulose (containing lignin in a large amount) as a first polysaccharide product to the cooler 3, by solid-liquid separation of a treated liquid coming from the reaction bath 1d, and delivers a polysaccharide containing a hemicellulose-derived oligosaccharide as a main component, as a second polysaccharide liquid, to the second catalytic reactor 7. The cooler 3 is installed to control the temperature of the first polysaccharide product such that the activity of a diastatic enzyme (heat-resistant enzyme) present in the enzymatic reactor 4 at the latter part becomes highest, and cools the first polysaccharide product coming from the solid-liquid separator 2 to, for example, about 50 to 90° C. and delivers it to the enzymatic reactor 4.

The enzymatic reactor 4 is an apparatus which adds a diastatic enzyme cellulase and water to the first polysaccharide product supplied from the cooler 3, and hydrolyzes cellulose into degradation product which contains cellobiose (glucose dimer) as a main component and is designated as a water soluble oligosaccharide (dimer to heptamer of glucose) or a suspended polysaccharide, through the action of cellulase on cellulose in the first polysaccharide product. Cellulase is generally known as an aggregate of plural diastatic enzymes, but contains β-glucanase as a main component. The β-glucanase is known as a diastatic enzyme for the hydrolysis of cellulose into cellobiose. Furthermore, the water soluble oligosaccharide is a water soluble degradation product (polysaccharide) as a dimer to hexamer of glucose, and the suspended polysaccharide is crystal of cellohexaose as a hexamer of glucose or a heptamer or more of glucose and exist as a suspended state in the enzymatic reactor 4. The enzymatic reactor 4 produces cellobiose through the action of the β-glucanase on cellulose, and delivers the first polysaccharide liquid containing the cellobiose as a main component to the first catalytic reactor 5.

Here, the diastatic enzyme used in the enzymatic reactor 4 may be one commercially available as a “heat-resistant enzyme”. A conventional diastatic enzyme exhibits a maximum enzymatic activity at a temperature of 40 to 50° C., whereas a heat-resistant enzyme exhibits a maximum enzymatic activity at a temperature of 70 to 90° C. Since a temperature range over which the cooler 3 must apply cooling is decreased through the use of such a heat-resistant enzyme in the enzymatic reactor 4, energy loss due to cooling of the first polysaccharide product can be reduced.

The first catalytic reactor 5 produces glucose through the hydrolysis of the first polysaccharide liquid discharged from the enzymatic reactor 4, using a powdered solid acid catalyst X, and delivers the first monosaccharide liquid containing the glucose as a main component to the first fermenter 6. As shown in the drawing, the first catalytic reactor 5 includes a first mixer 5a and a first solid-liquid separator 5b.

The first mixer 5a stirs and mixes the first polysaccharide liquid coming from the enzymatic reactor 4 and the previously filled solid acid catalyst X at a temperature of 90 to 100° C. to make contact therebetween, thereby promoting the hydrolysis reaction (i.e., saccharification reaction). By such a saccharification reaction, cellobiose contained in the first polysaccharide liquid is degraded to produce glucose which is a monosaccharide (hexose). The first monosaccharide liquid containing the thus produced glucose and the first mixture containing the solid acid catalyst X are discharged to the first solid-liquid separator 5b from the first mixer 5a.

The first solid-liquid separator 5b separates the first monosaccharide liquid from the solid acid catalyst X through solid-liquid separation of the first mixture coming from the first mixer 5a, recovers and supplies the solid acid catalyst X to the first mixer 5a (reuse), and delivers the glucose-containing first monosaccharide liquid to the first fermenter 6. As the first solid-liquid separator 5b, for example a sedimentation tank may be used. That is, the powdered solid acid catalyst X in the first mixture supplied to the sedimentation tank is precipitated at the bottom of the tank, and a supernatant is obtained as a first monosaccharide liquid containing glucose.

The first fermenter 6 adds ethanol fermentation microorganisms such as yeast and nutritive substances such as nitrogen and phosphorus to the glucose-containing first monosaccharide liquid coming from the first catalytic reactor 5, and produces ethanol from alcohol fermentation of glucose by the cultivation of microorganisms under optimum temperature and pH conditions, and the like. The first fermenter 6 delivers the ethanol thus produced to the distillation unit 9.

The second catalytic reactor 7 produces a second monosaccharide liquid containing a hemicellulose-derived monosaccharide through the hydrolysis of the second polysaccharide liquid coming from the pressurized hot water reactor 1, using a powdered solid acid catalyst X. As shown in the drawing, the second catalytic reactor 7 includes a second mixer 7a and a second solid-liquid separator 7b. The second mixer 7a stirs and mixes the second polysaccharide liquid coming from the pressurized hot water reactor 1 and the previously filled solid acid catalyst X at a temperature of 90 to 100° C. to make contact therebetween, thereby promoting the hydrolysis reaction (i.e., saccharification reaction). By such a saccharification reaction, a hemicellulose-derived oligosaccharide contained in the second polysaccharide liquid is hydrolyzed to produce a monosaccharide (pentose). The second mixer 7a delivers a second monosaccharide liquid containing the thus produced hemicellulose-derived monosaccharide and the second mixture containing the solid acid catalyst X to the second solid-liquid separator 7b.

The second solid-liquid separator 7b separates the second monosaccharide liquid from the solid acid catalyst X through solid-liquid separation of the second mixture coming from the second mixer 7a, recovers and supplies the solid acid catalyst X to the second mixer 7a (reuse), and delivers the hemicellulose-derived monosaccharide-containing second monosaccharide liquid to the second fermenter 8. As the second solid-liquid separator 7b, for example a sedimentation tank may be used as in the above-stated first solid-liquid separator 5b. That is, the powdered solid acid catalyst X in the second mixture supplied to the sedimentation tank is precipitated at the bottom of the tank, and a supernatant is obtained as a second monosaccharide liquid containing a hemicellulose-derived monosaccharide.

The second fermenter 8 adds ethanol fermentation microorganisms such as yeast and nutritive substances such as nitrogen and phosphorus to the hemicellulose-derived monosaccharide-containing second monosaccharide liquid coming from the second catalytic reactor 7, and produces ethanol from alcohol fermentation of the hemicellulose-derived monosaccharide by the cultivation of microorganisms under optimum temperature and pH conditions, and the like. As the ethanol-fermenting microorganism, it is possible to use various known microorganisms, such as yeast belonging to the genus Saccharomyces. The second fermenter 8 delivers the thus produced ethanol to the distillation unit 9.

The distillation unit 9 produces high-purity ethanol by performing distillation and concentration of ethanol coming from the first fermenter 6 and the second fermenter 8, and delivers the ethanol to the outside. The drainage unit 10 receives blow water discharged from the reaction bath 1d of the pressurized hot water reactor 1 and water (water produced during the alcohol fermentation process) discharged from the first fermenter 6 and the second fermenter 8, and discharges them to the outside after a given purification treatment.

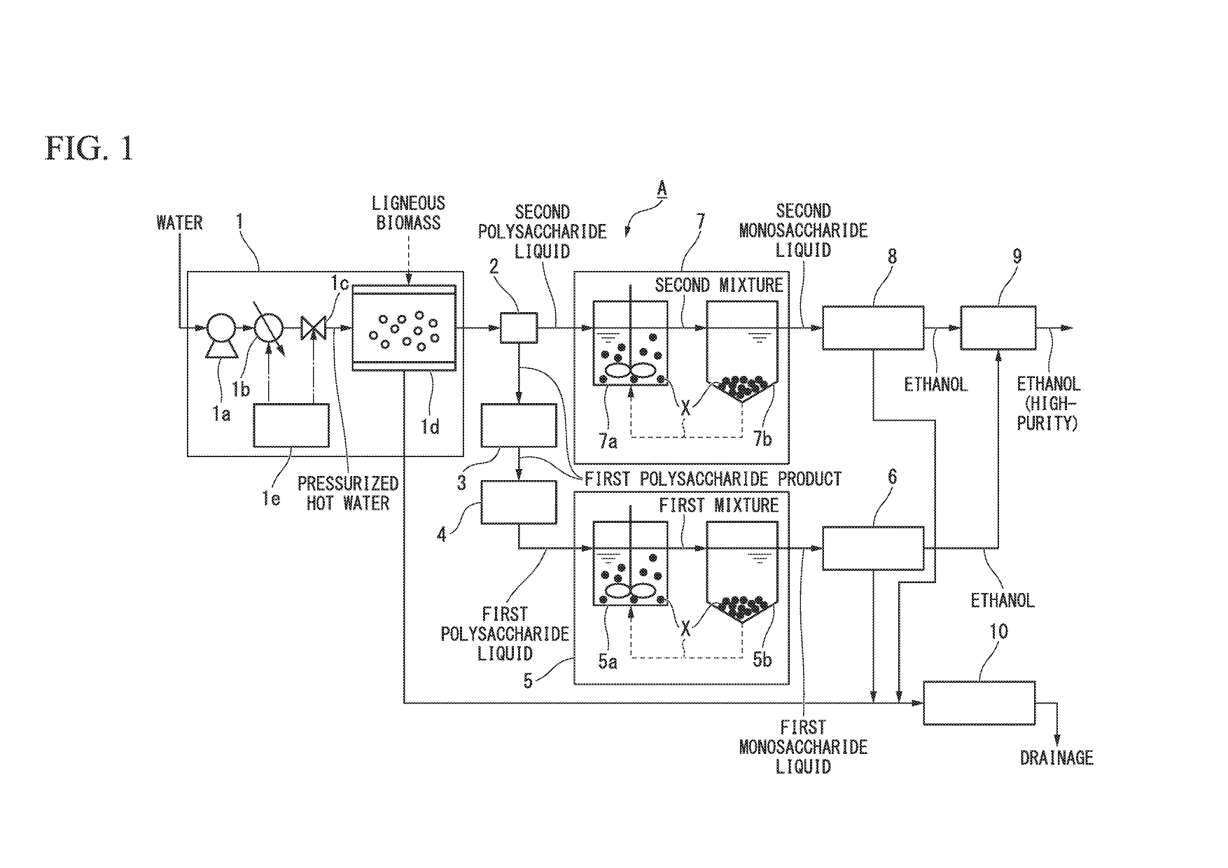

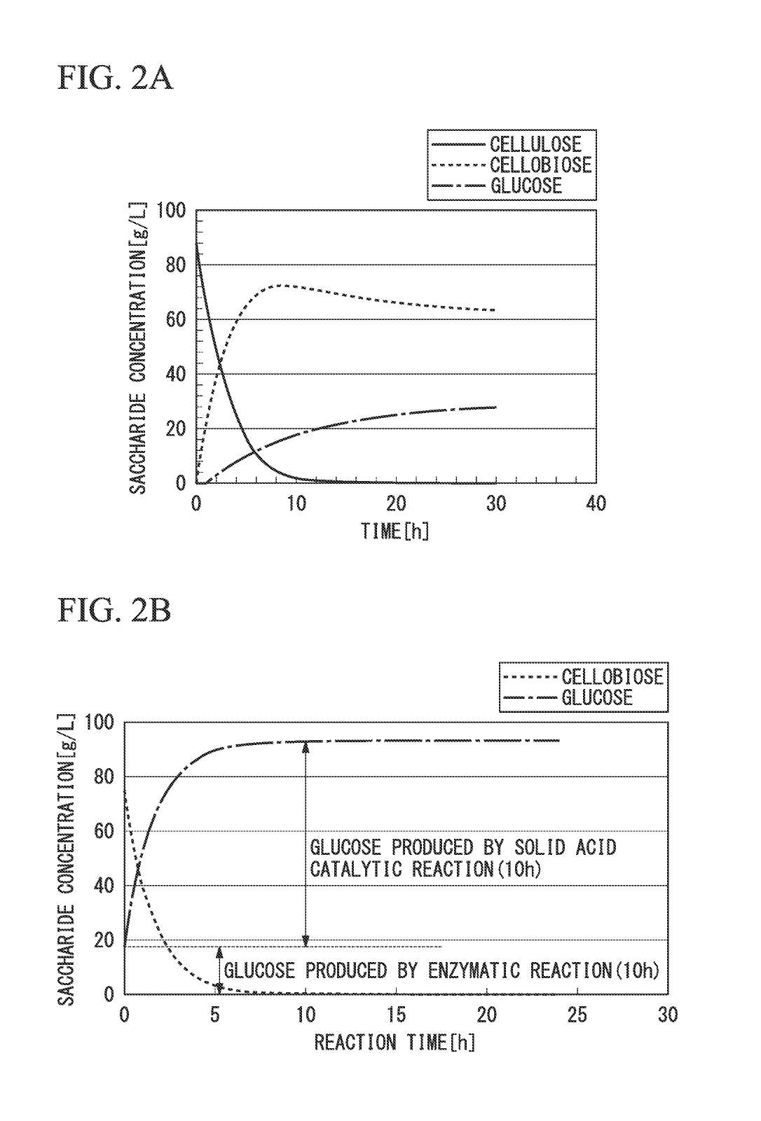

Next, the operation of the ethanol production apparatus A as thus configured will be described in more detail with reference to FIGS. 1, 2A and 2B.

In the pressurized hot water reactor 1, the heater 1b and the water volume control valve 1c are controlled by the control unit 1e, whereby pressurized hot water of 150 to 230° C., more preferably 200 to 230° C. is added in a given amount to a given amount of ligneous biomass accommodated in the reaction bath 1d. By allowing for a given reaction time to pass under such a state, hemicellulose contained in the ligneous biomass in the reaction bath 1d is selectively degraded into a polysaccharide (hemicellulose degradation product) containing a hemicellulose-derived oligosaccharide as a main component.

Accordingly, the treated liquid of the reaction bath 1d after the above-stated reaction time has passed becomes a solid-liquid mixture which contains cellulose and lignin as a solid and also contains a polysaccharide (hemicellulose degradation product) containing hemicellulose-derived oligosaccharide as a main component, as a liquid. Such a treated liquid is discharged to the solid-liquid separator 2 from the reaction bath 1d, and is subjected to solid-liquid separation in the solid-liquid separator 2. That is, cellulose and lignin as a solid are delivered as a first polysaccharide product to the cooler 3 from the solid-liquid separator 2, whereas the polysaccharide (hemicellulose degradation product) containing a hemicellulose-derived oligosaccharide as a main component is delivered as a second polysaccharide liquid to the second catalytic reactor 7.

The first polysaccharide product is cooled to a temperature of 90 to 100° C. in the cooler 3 and is delivered to the enzymatic reactor 4, and cellulase and water are added to the enzymatic reactor 4. As a result, through the action of cellulase on cellulose contained in the first polysaccharide product, the cellulose is degraded into cellobiose. That is, in the enzymatic reactor 4, β-glucanase contained in cellulase acts on cellulose to thereby produce cellobiose. In addition, cellobiose produced in the enzymatic reactor 4 is partially degraded into glucose through the action of β-glucosidase contained in cellulose.

FIG. 2A shows concentrations (g/L) of cellulose, cellobiose and glucose, when 1.5 g of cellulase is allowed to act on 1 L of the first polysaccharide product. As shown in FIG. 2A, most of the cellulose is degraded into cellobiose about 10 hours after initiation of the enzymatic reaction. Cellobiose is partially degraded into glucose, in conjunction with the production thereof. Based on indications shown in FIG. 2A, in the enzymatic reactor 4, the enzymatic reaction is carried out over the time until most of the cellulose is degraded into cellobiose. The resulting first polysaccharide liquid containing cellobiose and glucose is delivered to the first mixer 5a of the first catalytic reactor 5 from the enzymatic reactor 4.

The first mixer 5a stirs and mixes the first polysaccharide liquid coming from the enzymatic reactor 4 and the solid acid catalyst X at a temperature of 90 to 100° C. whereby cellobiose in the first polysaccharide liquid is degraded into glucose. The production rate of glucose in the first mixer 5a is higher than the production rate thereof in the enzymatic reactor 4. Specifically, the amount of glucose produced during about 10 hours in the first mixer 5a, as shown in FIG. 2B, is about 3.5 times the production amount of glucose in the enzymatic reactor 4. Based on indications shown in FIG. 2B, in the first mixer 5a, the catalytic reaction is carried out over the time until most of the cellobiose is degraded into glucose. The resulting first monosaccharide liquid containing glucose, together with the solid acid catalyst X, is delivered as a first mixture to the first solid-liquid separator 5b from the first mixer 5a.

The first mixture is solid-liquid separated into the first monosaccharide liquid and the solid acid catalyst X in the first solid-liquid separator 5b, and the solid acid catalyst X which is a solid is recycled to the first mixer 5a, whereas the first monosaccharide liquid is delivered to the first fermenter 6. In the first fermenter 6, ethanol is produced through alcohol fermentation from glucose contained in the first monosaccharide liquid, and is supplied to the distillation unit 9. In the distillation unit 9, distillation and concentration of ethanol are carried out.

On the other hand, with regard to the second polysaccharide liquid supplied to the second catalytic reactor 7, the second polysaccharide liquid and the solid acid catalyst X in the second mixer 7a of the second catalytic reactor 7 are stirred and mixed at a temperature of 90 to 100° C. whereby a hemicellulose-derived oligosaccharide in the second polysaccharide liquid is degraded into a monosaccharide. The second monosaccharide liquid containing a hemicellulose-derived monosaccharide, together with the solid acid catalyst X, is discharged as a second mixture to the second solid-liquid separator 7b from the second mixer 7a, and is solid-liquid separated into the second monosaccharide liquid and the solid acid catalyst X in the second solid-liquid separator 7b. The solid acid catalyst X which is a solid is recycled to the second mixer 7a, whereas the second monosaccharide liquid is delivered to the second fermenter 8. In the second fermenter 8, ethanol is produced through alcohol fermentation from the hemicellulose-derived monosaccharide contained in the second monosaccharide liquid, and is supplied to the distillation unit 9, followed by distillation and concentration.

Further, blow water discharged from the reaction bath 1d of the pressurized hot water reactor 1 and water (water produced during the alcohol fermentation process) discharged from the first fermenter 6 and the second fermenter 8 are discharged to the outside through the drainage unit 10 after cleaning with a purification treatment.

In such an ethanol production apparatus A, cellulose is degraded into cellobiose through the action of cellulase, further the cellobiose is degraded into glucose through the action of the solid acid catalyst X, and further, ethanol is produced from the glucose. That is, this ethanol production apparatus A performs the degradation of cellobiose into glucose using the reusable solid acid catalyst X in place of cellulase (more precisely, β-glucosidase contained in cellulase), so it is possible to saccharify cellulose in ligneous biomass while reducing cellulase costs, and it is also possible to produce ethanol from ligneous biomass while reducing cellulase costs.

Further, as shown in FIGS. 2A and 2B, since cellobiose is degraded into glucose using the solid acid catalyst X, it is possible to produce glucose at a higher rate as compared to the use of cellulase.

In this ethanol production apparatus A, since a temperature range over which the cooler 3 must apply cooling is decreased by using a heat-resistant enzyme in the enzymatic reactor 4, energy loss due to cooling of the first polysaccharide product can be reduced. That is, the first polysaccharide product is maximally heated to 200 to 230° C. in the pressurized hot water reactor 1, and upon using a conventional diastatic enzyme, the temperature of the first polysaccharide product should be lowered to about 70° C. at which an enzymatic activity becomes highest, but lowering the temperature of the first polysaccharide product to about 90 to 100° C. is sufficient when using a heat-resistant enzyme, so energy loss can be reduced.

Further, by the use of a heat-resistant enzyme, the reaction temperature of the enzymatic reactor 4 can be made equal to the reaction temperature of the first mixer 5a. Accordingly, energy efficiency can be improved due to not needing heating in the first mixer 5a.

### ADDITIONAL DISCLOSURES

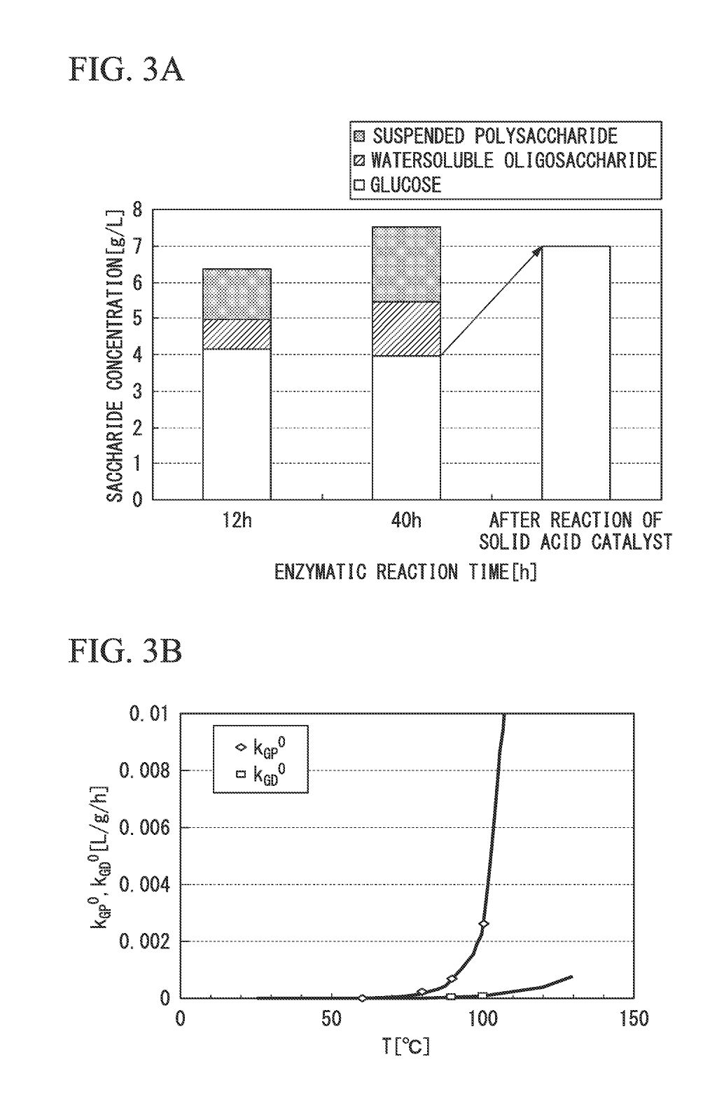

FIG. 3A is a bar diagram which shows concentration of degradation products in accordance with an embodiment of the present invention based on experimental results. In FIG. 3A, bar diagrams of left side and center denote concentration of degradation products in the first polysaccharide liquid obtained from the enzymatic reactor 4 respectively, and the bar diagram of right side denotes concentration of degradation products in the first polysaccharide liquid obtained from the first catalytic reactor 5. Furthermore, the bar diagram of left side denotes the case in which the reaction time in the enzymatic reactor 4 is 12 hours, and the bar diagram of center denotes the case in which the reaction time in the enzymatic reactor 4 is 40 hours.

According to the above experiments, it is recognized that the above-described suspended polysaccharide, water soluble oligosaccharide and glucose are obtained as the degradation product, and the suspended polysaccharide and the water soluble oligosaccharide and glucose in the degradation product are degraded into glucose (monosaccharide) in the first catalytic reactor 5.

FIG. 3B is a line graph which shows temperature dependency of a production rate constant of glucose k_{GP}⁰ and a decomposition constant of glucose k_{GD}⁰ of the solid acid catalyst X in accordance with an embodiment of the present invention based on experimental results. According to the above experiments, it is recognized that the reaction temperature in the first and second catalytic reactors 5, 7 is preferably set to 90° C. or higher and to lower than 120° C.; because the reaction temperature in order to obtain glucose by the solid acid catalyst X is preferably set to 90° C. or higher, and even when the temperature is higher than 100° C., degradation to glucose does not increase extremely as far as the temperature is lower than 120° C.

The present invention is not limited to the above-stated embodiments. For example, the following variants are conceived.

(1) In the above-stated embodiments, although ethanol is produced from glucose produced in the first catalytic reactor 5 or from hemicellulose-derived monosaccharide produced in the second catalytic reactor 7, the present invention is not limited thereto. For example, chemical products (for example, hydroxymethylfurfural or furfural) other than ethanol may be produced by replacing the first fermenter 6, or the second fermenter 8, or the distillation unit 9 with another reactor.

Furthermore, in the above-stated embodiments, although yeast belonging to the genus Saccharomyces is used as the ethanol-fermenting microorganism, lactic fermentation by lactic acid bacteriumin or butanol fermentation by bacteria belonging to the genus Clostridium may be performed in the first fermenter 6 and second fermenter 8 in order to obtain lactic acid or buthanol as the fermentative products, for example.

That is, the fermentation apparatus according to the present invention is not limited to the fermentation apparatus for producing ethanol, and includes the fermentation apparatus for producing other fermentative products.

(2) In the above-stated embodiments, although glucose is produced from cellulose contained in ligneous biomass, the present invention is not limited thereto. Glucose may be produced from cellulose contained in herbaceous biomass, or from artificially produced cellulose.

(3) In the above-stated embodiments, although a heat-resistant enzyme is used in the enzymatic reactor 4, the present invention is not limited thereto. For example, it may be configured such that the cooler 3 cools the second polysaccharide product containing cellulose to about 50° C., and cellulose is degraded into cellobiose in the enzymatic reactor 4 using a conventional enzyme whose enzymatic activity is optimal at about 50° C. When a conventional enzyme is used in such a manner, the reaction temperature of hydrolysis by the solid acid catalyst X at the latter part is preferably set to the reaction rate (i.e. about 50° C.) in the enzymatic reactor 4, from the viewpoint of energy efficiency. However, since 50° C. is a reaction temperature which is lower than 90 to 100° C. in the case of using a heat-resistant enzyme, the reaction rate of hydrolysis by the solid acid catalyst X decreases. In order to compensate for such a decrease in reaction rate, it is considered that the amount of the solid acid catalyst X is increased as compared to when a heat-resistant enzyme is used.

(4) In the above-stated embodiments, although cellulase is reacted with the first polysaccharide product containing cellulose and lignin in the enzymatic reactor 4, the present invention is not limited thereto. For example, it may be configured such that a process of removing lignin from the first polysaccharide product is provided at the former part of the enzymatic reactor 4, and the lignin-removed first polysaccharide product is supplied to the enzymatic reactor 4. Thereby, the concentration of a diastatic enzyme in the first polysaccharide product in the enzymatic reactor 4 is enhanced, so the degradation rate of cellulose can be increased.

(5) In the above-stated embodiments, since the first catalytic reactor 5 and the second catalytic reactor 7 employ a powdered solid acid catalyst X, the first solid-liquid separator 5b and the second solid-liquid separator 7b are provided. However, the solid acid catalyst may be a pellet-like catalyst other than the powder type. When the pellet-like solid acid catalyst is used, it is considered that as the first catalytic reactor and the second catalytic reactor, for example a catalytic reactor (fixed-bed solid acid catalytic reactor) is adopted of a type in which hydrolysis is carried out by passing the first polysaccharide liquid or second polysaccharide liquid through a pellet-like solid acid catalyst received in a fixed state in a circulatory container. By employing such a fixed-bed solid acid catalyst reactor, the configuration of the first catalytic reactor and the second catalytic reactor can be simplified.

### INDUSTRIAL APPLICABILITY

According to the present invention, diastatic enzyme costs can be further reduced as compared to the conventional art, even when fermentable sugar such as glucose is produced from cellulose contained in biomass, and further even when fermentable sugar such as glucose is produced from cellulose contained in biomass and fermentative products such as ethanol is produced from the fermentable sugar.

### EXPLANATION OF REFERENCES

A: ethanol production apparatus (fermentation apparatus),
1: pressurized hot water reactor,
1a: pump,
1b: heater,
1c: water volume control valve,
1d: reaction bath,
1e: control unit,
2: solid-liquid separator,
3: cooler,
4: enzymatic reactor,
5: first catalytic reactor,
5a: first mixer,
5b: first solid-liquid separator,
6: first fermenter,
7: second catalytic reactor,
7a: second mixer,
7b: second solid-liquid separator,
8: second fermenter,
9: distillation unit,
10: drainage unit.

1. A cellulose saccharification apparatus, comprising:
an enzymatic reactor for degrading cellulose using a diastatic enzyme, and
a first catalytic reactor for degrading the degradation product produced by the enzymatic reactor into glucose, using a solid acid catalyst.

2. The cellulose saccharification apparatus according to claim 1, wherein the diastatic enzyme is a heat-resistant enzyme.

3. A biomass saccharification apparatus, comprising:
a pressurized hot water reactor for selectively degrading hemicellulose contained in biomass by allowing pressurized hot water to act on the biomass,
a solid-liquid separator for separating cellulose as a solid from a treated liquid of the pressurized hot water reactor, and
a cellulose saccharification apparatus of claim 1 for degrading cellulose separated by the solid-liquid separator into glucose.

4. The biomass saccharification apparatus according to claim 3, further comprising a second catalytic reactor for degrading a hemicellulose degradation product as the liquid separated by the solid-liquid separator, into a hemicellulose-derived monosaccharide, using a solid acid catalyst.

5. A fermentation apparatus, comprising:
the biomass saccharification apparatus of claim 4,
a first fermenter for producing fermentative products from glucose produced by the biomass saccharification apparatus, and
a second fermenter for producing fermentative products from a hemicellulose-derived monosaccharide produced by the biomass saccharification apparatus.

6. A cellulose treatment method, comprising:
an enzymatic reaction process for degrading cellulose using a diastatic enzyme, and a solid acid catalytic reaction process for degrading the degradation product produced by the enzymatic reaction process into glucose, using a solid acid catalyst.
